(19) United States
(12) Patent Application Publication
(10) Pub. No.: US 2004/0097429 A1
(43) Pub. Date: May 20, 2004

(54) Method for the reduction of the mammalian appetite

(76) Inventors: Arie Gijsbert Nieuwenhuizen, Utrecht (NL); Rene John Raggers, Amsterdam (NL); Hobbe Friso Smit, Utrecht (NL)

Correspondence Address: YOUNG & THOMPSON, 745 SOUTH 23RD STREET 2ND FLOOR, ARLINGTON, VA, 22202

(21) Appl. No.: 10/295,987

(22) Filed: Nov. 18, 2002

(51) Int. Cl. A61K 31/7048; A61K 31/353

(52) U.S. Cl. 514/27; 514/456

(57) ABSTRACT

The present invention relates to a method for the treatment and/or prevention of overweight and to a method for the reduction of a mammalian appetite. The present method comprises the administration to the mammal of a dosage containing an effective amount of a flavonoid selected from the group consisting of chrysin, flavone, precursors of these flavonoids that are convertible into the one of these flavonoids by gastrointestinal hydrolytic cleavage and mixtures thereof.

### BACKGROUND OF THE INVENTION

Overweight and obesity are major problems within the Western community. Due to increased consumption, decreased exercise and changes in the nutritional value of foodstuffs, many humans and companion animals are suffering from overweight or have difficulty maintaining a desirable weight. Many methods have been proposed to solve this problem, for example, via the administration of functional ingredients (e.g. nutritional supplements) which facilitate the reduction of overweight.

In effect, overweight is caused by the ingestion of excess calories. Calories are for example ingested via high caloric meals. Within the art, several strategies are known for reducing caloric intake. These strategies include for example replacing the high caloric meals for low caloric meals (e.g. meal replacers); the ingestion of medicaments that reduce the absorption of high caloric components from the meal (e.g. lipase inhibitors); and the administration of appetite reducing agents.

Presently known appetite reducing agents have the disadvantage that they cause severe side effects. Sibutramine is an appetite suppressant that is proposed to work via norepinephrine and serotonergic mechanisms in the brain. The drug has been described to have side effects, including high blood pressure, headache, dry mouth (which may increase the risk for dental disease), constipation and decreased sleep. Other uncommon side effects include: increased heart rate, dizziness, flushing, sweating, nausea. Presently, sibutramine is the only appetite suppressant that has been approved by the FDA for long-term use in a method for reducing overweight.

The present inventors have surprisingly found that particular flavonoids such as chrysin are capable of reducing the appetite when administered orally. Chrysin (5,7-dihydroxyflavone) is a flavonoid that is advertised to promote muscle growth. Furthermore, chrysin has been described to possess anxiolytic properties (i.e. anxiety reducing properties) without exhibiting a sedative effect (U.S. Pat. No. 5,756,538). Chrysin has also been described to treat any condition of elevated levels of unconjugated bilirubin in adults or children, such as Gilbert's syndrome or liver cirrhosis (WO0158410). WO9922728 relates to compounds that inhibit 5 alpha -reductase. The compounds are used to treat prostate cancer, breast cancer, obesity, skin disorders and baldness. From Table 1 in the document it is apparent that chrysin is unsuitable as a compound to inhibit 5 alpha reductase, hence unsuitable to treat prostate cancer, breast cancer, obesity, skin disorder and baldness.

In addition, chrysin has been incorporated in products designed to increase muscle growth. These products contain androgens, anabolic steroids and/or prohormones, An example of such a product is Cycloroid™. The chrysin incorporated in this product aims to reduce the side effects resulting from the administration of the androgens, anabolic steroids or prohormones, e.g. the formation of fat deposits. The present invention does not relate to the use of chrysin in combination with androgens, anabolic steroids and/or prohormones.

### SUMMARY OF THE INVENTION

The present inventors surprisingly found that chrysin and flavone are capable of reducing the mammalian appetite without significant side effects.

One aspect of the present invention relates to a method for reducing the mammalian appetite, comprising the administration to a mammal of an effective amount of a flavonoid selected from the group consisting of chrysin, flavone, precursors of these flavonoids that are convertible into the one of these flavonoids by gastrointestinal hydrolytic cleavage and mixtures thereof.

Reduction of appetite results in a reduced caloric intake. Since excessive caloric intake is a main cause of overweight, the aforementioned flavonoids can be advantageously used to reduce overweight. Hence, in a further aspect the present invention relates to a method for the treatment and/or prevention of overweight and obesity, said method comprising the administration of an effective amount of one or more of the above mentioned flavonoids. Chrysin was found to be particularly suitable for use in a method for the prevention and/or reduction of overweight and appetite.

### DETAILED DESCRIPTION OF THE PREFFERED EMBODIMENTS

The present invention provides a method for the treatment or prevention of overweight in a mammal, said method comprising the oral administration to the mammal of a dosage containing an effective amount or a flavonoid selected from the group consisting of chrysin, flavone, precursors of these flavonoids that are convertible into the one of these flavonoids by gastrointestinal hydrolytic cleavage and mixtures thereof, said dosage being substantially free of androgens, anabolic steroids and/or prohormones.

Preferably, the precursors of the flavonoids that are convertible into one of these flavonoids by gastrointestinal hydrolytic cleavage are selected from the group consisting of glycosides, rutinosides, glucuronoside, gentobioside and methyl ethers, more preferably glycosides.

Advantageously chrysin or a glycoside thereof is used. Preferred glycosides of chrysin are selected from the group consisting of chrysin-5-O-glycoside and chrysin-7-O-glycoside.

In a further aspect, the present invention provides a method for the reduction of appetite in a mammal, said method comprising the oral administration to the mammal of a dosage containing an effective amount of a flavonoid selected from the group consisting of chrysin, flavone, precursors of these flavonoids that are convertible into the one of these flavonoids by gastrointestinal hydrolytic cleavage and mixtures thereof. Preferably the flavonoid is selected from the group consisting of chrysin, flavone and glycosides thereof, more preferably chrysin or a glycoside thereof is used.

Dosage

In order to achieve significant weight reduction and/or appetite reduction, it is desirable to ingest at least 0.01 mg, preferably at least 0.1 mg, even more preferably at least 1 mg, most preferably at least 2 mg of the present flavonoid per kg of body weight per dosage. A dosage as used in the present method preferably contains less than 250 mg, more preferably less than 100 mg, even more preferably less than 75 mg, most preferably less than 50 mg of the present flavonoid per kg of body weight. Hence, for a human adult the present method preferably comprises the administration of a dosage containing between 5 mg and 10 grams, more preferably between 25 mg and 5 grams, even more preferably between 50 mg and 3 grams, most preferably between 100 mg and 1.5 grams of the flavonoid, preferably chrysin. Whenever the term dose or dosage is used within this disclosure, any dosage form is encompassed which can be administered, preferably orally, within a fairly narrow time span. Whenever reference is made to a certain quantity that is administered per dose or dosage, said quantity is preferably administered within one hour, more preferably within 15 minutes, even more preferably within 5 minutes.

Sources

In a particularly preferred embodiment, the flavonoid, preferably chrysin and glycosides thereof, is provided by a flavonoid containing plant material, more preferably in the form of a plant isolate. The term “plant isolate” as referred to in here, encompasses any fraction that can be obtained from a plant material by means of isolation techniques known in the art, e.g. extraction, distillation, squeezing etc. and that has an increased flavonoid content compared to the dry plant raw material. Preferably the plant material contains at least 1 wt. %, more preferably at least 5 wt. %, even more preferably at least at least 10 wt. %, most preferably at least 50 wt. % flavonoid based on the total dry weight of the plant material. Usually, the flavonoid content of the plant material does not exceed 99.5 wt. % based on the total dry weight of the plant raw material.

According to a preferred embodiment the present flavonoid is provided in the form of a plant material or extract from a plant selected from the group consisting of Pinus aristata, Prunus domestica, Ulmus sieboldiana, Fluorensia resinosa, Oroxylum indicum, Scutellaria spp., Passiflora spp. and mixtures thereof, more preferably form a plant selected from the group consisting of Pinus aristata, Prunus domestica, species of Passiflora and mixtures thereof, most preferably selected from plant material or isolate from a plant selected from the group consisting of Passiflora alata, Passiflora incarnata, Passiflora coerulea and mixtures thereof.

Purity

Advantageously, the present method comprises the administration of a dosage containing at least 1 wt. %, more preferably at least 5 wt. %, even more preferably at least 10 wt. %, most preferably at least 25 wt. % of the present flavonoid based on the total dry weight of the dosage. To reduce dosage size, preferably the chrysin is provided by a composition containing at least 25 wt. %, more preferably containing at least 50 wt. %, even more preferably at least 90 wt. % one of the present flavonoids. Advantageously, synthetic chrysin is used.

Treatment and Prevention of Overweight

The present invention provides a method for reducing or preventing overweight or obesity, preferably a method for the reduction the appetite in a mammal. The term overweight as used in the present invention refers to a bodyweight that is above the desired bodyweight. The present method is particularly suitable for humans. Human subjects who have a body mass index above 25 most advantageously use the present method.

Packaged nutritional supplements and dietary products, which have been provided with labels that explicitly or implicitly direct the consumer towards the use of said supplement or product in accordance with one or more of the above or below purposes, are encompassed by the present invention. Such labels may for example make reference to the method for the treatment of overweight by incorporation of terminology like “slim”, “lean”, “weight reduction” and the like. The overweight reducing properties of the product may be indicated via indicia such as pictures, drawings and other indicia from which a consumer can conclude that the product aims to treat or prevent overweight. The present invention does not relate to the use of chrysin for counteracting side effects of the androgens, anabolic steroids or prohormones. Androgens, anabolic steroids and prohormones are often used to increase muscle mass, and thus body weight. In a preferred embodiment the present method does not comprise the administration of a member selected from the group consisting of an androgens, anabolic steroids and prohormones, i.e. comprises the administration of a dosage free of androgens, anabolic steroids and prohormones. In other words, the present method preferably does not comprise the administration of any androgens, anabolic steroids or prohormones. The term “prohormones” as used in the present invention relates metabolic precursors which raise the level of the male hormones testosterone and/or 19-nortestosterone in vivo, preferably to those ingredients that are metabolic precursors of hormones.

Reduction of appetite

The present invention is especially aimed at the reduction or prevention of appetite and/or feelings of hunger. The method according to the invention can, for example, be used in a method for inducing satiety, inducing satiation, satisfying hunger or reducing craving urges.

Generally, an individual's feelings and sensations between the start of a first meal and the next meal go through different phases. A set of sensations is usually discriminated within the art. If satiety is evaluated, several phases can be used to express the satiety after a meal. These can be termed very full, full, appetite and hungry. Preferably the flavonoid is administered in the phases appetite, hunger or at the end of the full phase, more preferably in the appetite or hunger phase.

Preferably, the present flavonoid is administered about 1-8 hours, more preferably about 2-6 hours after consumption of a meal. Typically the flavonoid is administered between 1 hour after one meal and 1 hour prior to the next meal. In a further aspect, the present flavonoid, preferably chrysin, can be taken shortly before the meal or even during a meal, for example when the meal is expected to provide insufficient satisfaction. This may occur when the subject is subjected to a weight loss program. Hence, the present flavonoid can be advantageously used in a method for the reduction of the adverse side effects experienced during a weight loss program, i.e. administered to subjects participating in a weight loss program.

Additionally, appetite reducing agents are useful in several other applications. The present flavonoids can for example be used to provide comfort to subjects having limited access to foodstuffs, such as for example military personal during a long mission.

Administration

According to a preferred embodiment the present method comprises the oral administration of a dosage. The dosage used in the present method can be applied in any suitable form, such as bars, pills, capsules, gels, liquid etc, however is preferably provided in the form of a pill, tablet or capsule. Preferably a dosage does not consist of more than 3 tablets, capsules or pills, even more preferably consists of a single pill, capsule or tablet. Advantageously, at least two dosages, more preferably at least three dosages, are administered in one day. In the present method a daily dosage of the preparation as used in the present invention can include one or more pills, tablets or capsules. Preferably a daily dosage consists of 1 to 6 pills, tablets or capsules.

A dosage is preferably in a solid or semisolid form, more preferably in a form selected from the group consisting of pills, capsules, tablets, caplet, microparticles and microspheres. The solid or semisolid dosage form preferably has a weight between 0.1 and 30 grams, more preferably between 0.2 and 10 grams.

The dosage preferably has a caloric value below 100 kcal, more preferably below 50 kcal even more preferably below 10 kcal. A dosage preferably has a weight between 0.2 and 4 grams, even more preferably between 0.5 and 3 grams.

### EXAMPLES

### Example 1

Chrysin

A: Appetite reducing effects of chrysin

The appetite suppressive effects of chrysin (Technical Sourcing International Inc) was tested in adult male Wistar rats. In a placebo-controlled cross-over study, either placebo or a single dosages of chrysin (50 mg/kg) were administered as a single bolus intragastrically at 30 min before onset of the active (dark) period. Subsequently, cumulative voluntary food intake was recorded continuously for 48 hours. Following this period, the experiment was repeated as part of the cross-over design (that is, rats which first received chrysin now received the placebo and vice versa).

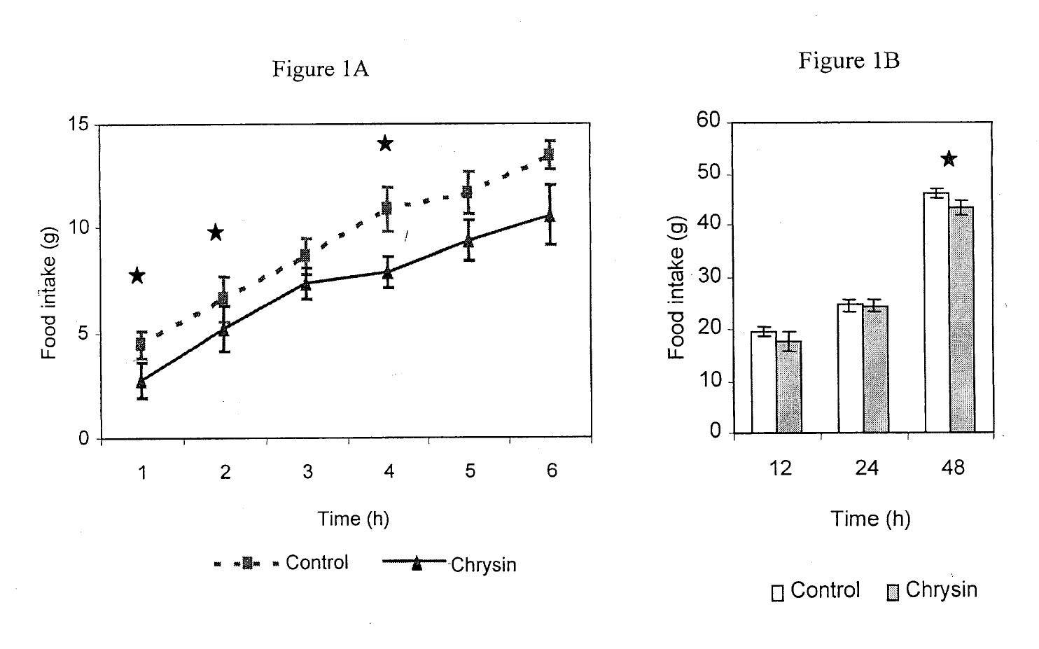

Results are shown as cumulative food intake. FIG. 1A shows reduced food intake compared to placebo. A dose of 50 mg/kg chrysin per kg rat resulted in a statistically significant (p<0.05) reduction of food intake at 1, 2 and 4 hours, indicating a reduced appetite. FIG. 1B shows the cumulative food intake over a period of 12, 24 and 48 hours after administration of chrysin. A dose of 50 mg chrysin per kg rat resulted in a statistically significant (p<0.05) reduction of food intake at 48 hours, indicative for the appetite reducing effect of chrysin.

B: Taste aversion

To exclude the possibility that the appetite reducing effects of chrysin is caused by the induction of discomfort or sickness, the effective dosage of chrysin was tested on the induction of taste aversion. The taste aversion test consisted of an acquisition and a testing period. During the acquisition period, the rats received, on each day, a small amount (3 ml) of custard with a specific taste selected from vanilla, chocolate or raspberry. Each taste was administered for 3 days. Immediately after complete consumption of one custard with a specific taste, 1 ml of a vehicle with or without a test compound was administered intragastrically. Three different test compounds were used: 1) vehicle alone (suspension gel), 2) vehicle containing chrysin (50 mg/kg body weight), and 3) lithium chloride (8 mg/kg body weight). Lithium chloride (LiCl) at this dosage is known to induce mild discomfort (nausea) and taste aversion, and is used as a positive control. Thus, in each rat for each period of 3 days, 1 specific taste of custard is associated with the administration of 1 compound. In this way, after the full acquisition period each of the 3 different custards were assigned to a different compound (custard compound-association). The specific custard-compound association was randomized between the animals in order to exclude non-acquired preference for a particular taste. In the testing period, each rat receives on each day all of the 3 custards with different taste at the same time, by placing the custards (in random order) in the cage. No test compounds are administered. Compound-induced taste aversion is measured as the percentage of rats leaving the custard associated with that particular compound untouched. Hence, the test is based on the assumption that a rat will develop an aversion for the taste custard that, in the acquisition period, was associated with a negative event (i.e. intragastric administration of a test compound that induces discomfort).

The test compound of which the associated custard was consumed first received 3 points, the second 2, and the third 1 point. In 8 animals, the maximal possible score for a compound amounts to 8×3=24 points, which was set to 100%. The aversion percentage of one compound was determined by dividing the measured score by 24 and multiplication with 100%.

Results showed a significant aversion towards the LiCl, while chrysin and placebo did not show any taste-aversion. Hence, the appetite reducing effects of chrysin is not caused by the induction of discomfort.

### Example 2

Appetite reducing effect of flavone

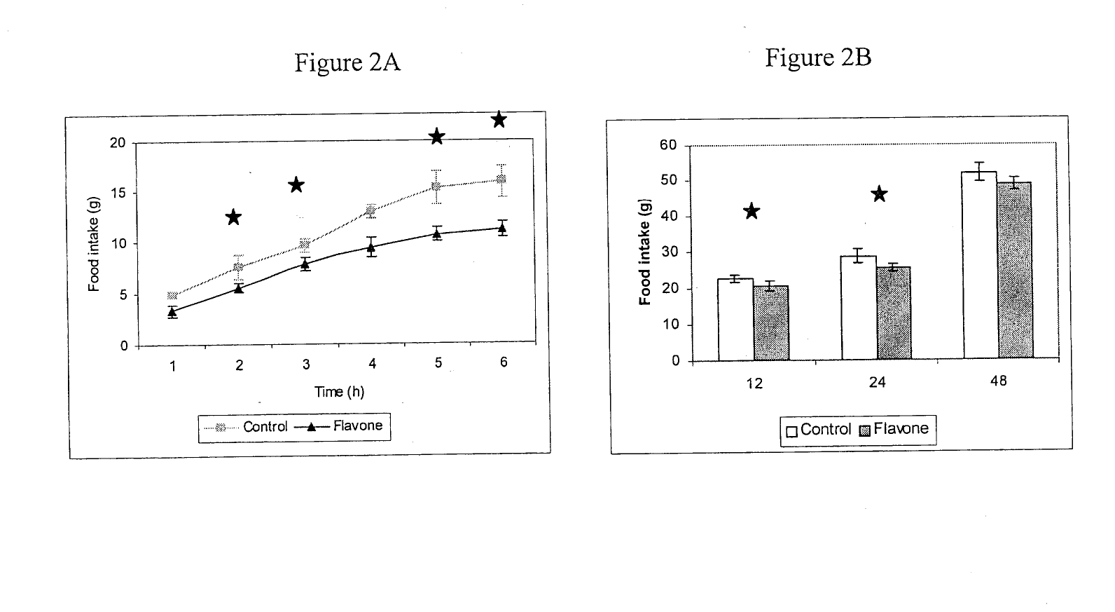

The appetite suppressive effects of flavone was tested in adult male Wistar rats in a method as described in Example 1A, with the difference that instead of administering 50 mg chrysin per kg chrysin, 25 mg flavone per kg was administered to the rats. Results are shown as cumulative food intake. FIG. 2A shows reduced food intake compared to placebo. A dose of 25 mg flavone per kg rat resulted in a statistically significant (p<0.05) reduction of food intake at 2, 3, 5 and 6 hours, indicating a reduced appetite. FIG. 2B shows the cumulative food intake over a period of 12, 24 and 48 hours after administration of flavone. A dose of 25 mg flavone per kg resulted in a statistically significant (p<0.05) reduction of food intake at 12 and 24 hours, indicative for the appetite reducing effect of flavone.

### Comparative Example 3

Appetite reducing effect op catechin

The appetite suppressive effects of catechin was tested in adult male Wistar rats in a method as described in Example 1A, with the difference that instead of administering 50 mg/kg chrysin, 40 mg/kg green tea extract (Nutratech, Faifield (N.J.); GT00043; green tea extract powder (80%) containing 70 wt. % catechins, including epigallocatechin gallate) was administered to the rats. 40 mg green tea per kg rat corresponds with about 28 mg catechin per kg rat.

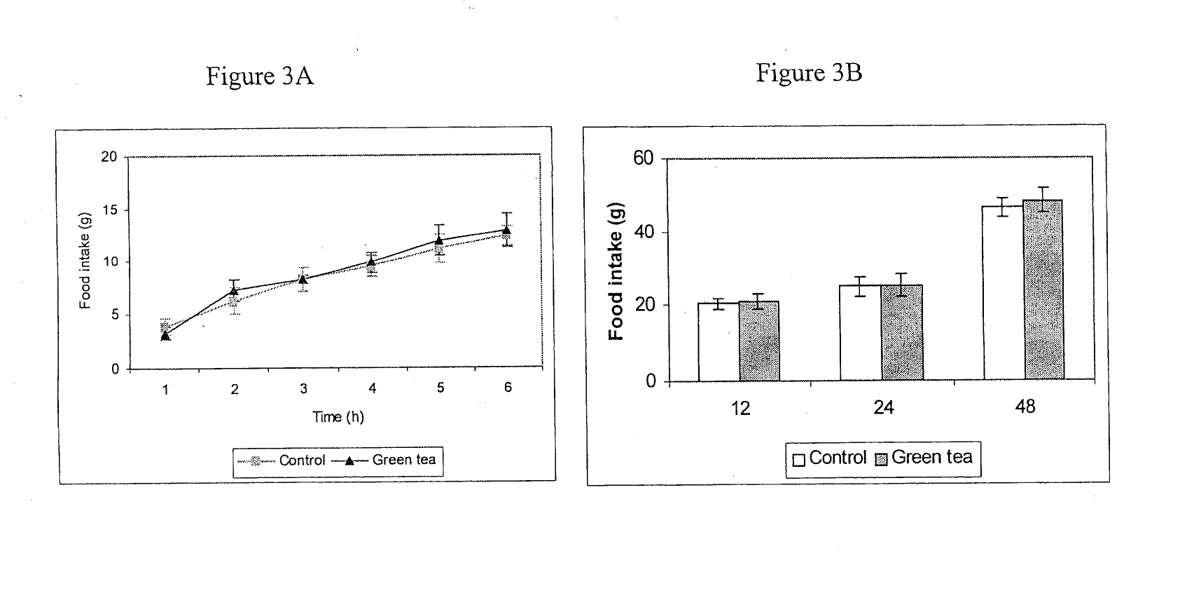

Results are shown as cumulative food intake. FIG. 3A shows no reduced food intake compared to placebo. FIG. 3B shows the cumulative food intake over a period of 12, 24 and 48 hours after administration of green tea extract. A dose of 40 mg green tea extract per kg did not result in a reduction of food intake.

### Example 4

Tablet containing chrysin

Tablet containing:

250 mg chrysin

250 mg excipient

1. A method for the treatment and/or prevention of overweight in a mammal, said method comprising the administration to the mammal of a dosage containing an effective amount of a flavonoid selected from the group consisting of chrysin, flavone, precursors of these flavonoids that are convertible into the one of these flavonoids by gastrointestinal hydrolytic cleavage and mixtures thereof, said dosage being substantially free of androgens, anabolic steroids and/or prohormones.

2. Method according to claim 1, wherein the flavonoid is chrysin.

3. Method according to claim 1, wherein the flavonoid is flavone.

4. Method according to claim 1, wherein the method comprises the administration of a dosage containing between 0.01 mg and 250 mg flavonoid per kg of body weight.

5. Method according to claim 1, wherein the method comprises the administration of a dosage containing between 5 mg and 10 grams of the flavonoid based on the total dry weight of the dosage.

6. Method according to claim 1, wherein the method comprises the administration of a dosage containing at least 1 wt. % of the flavonoid.

7. Method according to claim 1, wherein the flavonoid is provided by a plant isolate.

8. Method according to claim 7, wherein the plant isolate contains at least 1 wt. % of the flavonoid based on the total dry weight of the plant isolate.

9. Method according to claim 1, wherein the mammal is a human.

10. Method according to claim 1, wherein the flavonoid is administered orally.

11. A method for the reduction of appetite in a mammal, said method comprising the oral administration to a mammal of a dosage containing an effective amount of a flavonoid selected from the group consisting of chrysin, flavone, precursors of these flavonoids that are convertible into the one of these flavonoids by gastrointestinal hydrolytic cleavage and mixtures thereof.

12. Method according to claim 11, wherein the flavonoid is chrysin.

13. Method according to claim 11, wherein the flavonoid is flavone.

14. Method according to claim 11, wherein the method comprises the administration of a dosage containing between 5 mg and 10 grams of the flavonoid based on the total dry weight of the dosage.

15. Method according to claim 1, wherein the method comprises the administration of a dosage containing at least 1 wt. % of the flavonoid.

16. Method according to claim 1, wherein the flavonoid is provided by a plant isolate.

17. Method according to claim 1, wherein the mammal is a human.

18. Method according to claim 1, wherein the flavonoid is administered orally.
